(19) United States
(12) Patent Application Publication
(10) Pub. No.: US 2024/0041719 A1
(43) Pub. Date: Feb. 8, 2024

(54) TEETH WHITENING COMPOSITIONS, KITS, AND SYSTEMS

(71) Applicant: ZEST IP HOLDINGS, LLC, Carlsbad, CA (US)

(72) Inventors: Yuping LI, Irvine, CA (US); Nicholas ARALIS, San Juan Capistrano, CA (US); Wen GU, Riverside, CA (US)

(21) Appl. No.: 18/379,052

(22) Filed: Oct. 11, 2023

Related U.S. Application Data
(63) Continuation of application No. 17/578,303, filed on Jan. 18, 2022.
(60) Provisional application No. 63/272,470, filed on Oct. 27, 2021.
(60) Provisional application No. 63/271,038, filed on Oct. 22, 2021.

(51) Int. Cl. A61K 8/22; A61K 8/81; A61K 8/34; A61K 8/21; A61K 8/04; A61Q 11/00; A61K 8/90

(52) U.S. Cl. CPC A61K 8/22; A61K 8/8182; A61K 8/345; A61K 8/21; A61K 8/042; A61Q 11/00; A61K 8/90; A61K 2800/88; A61K 2800/48; A61K 2800/594

(57) ABSTRACT

Dental whitening compositions, kits and systems exhibiting stability in viscosity and hydrogen peroxide concentration for an extended shelf life without refrigeration or other special storage conditions are provided herein. The composition may be in gel form and comprise a hydrogen peroxide or a hydrogen peroxide precursor, and a thickening agent comprising a vinylpyrrolidone polymer or copolymer. In some aspects, a kit may be provided comprising a two-part dental whitening system, wherein one part comprises a whitening agent, and a separate part comprises a strong base.

### CROSS-REFERENCE TO RELATED APPLICATION

This application is a continuation application of U.S. application Ser. No. 17/578,303, filed Jan. 18, 2022, which claims priority to U.S. Provisional Application No. 63/271,038, filed on Oct. 22, 2021 and 63/272,470, filed on Oct. 27, 2021. These and all other extrinsic materials discussed herein, including publications, patent applications, and patents, are incorporated by reference in their entirety. Where a definition or use of a term in an incorporated reference is inconsistent or contrary to the definition of that term provided herein, the definition of that term provided herein applies and the definition of the term in the reference does not apply.

### FIELD OF THE INVENTION

The field of the invention is teeth whitening compositions, kits, and systems.

### BACKGROUND

Teeth can become discolored as a consequence of tobacco use, hemorrhages or disorders during odontogenesis, or intake of certain medications. Teeth whitening is often desirable for aesthetic purposes, and in recent decades, teeth whitening has become increasingly popular, whether through dental office visits or certain at-home kits. Teeth whitening involves an application of peroxide containing gel or paste compositions to the surface of the teeth or from the inside of teeth (e.g., endodontic bleaching).

### SUMMARY

In some aspects, a tooth whitening composition is provided, comprising at least one whitening agent, at least one thickening agent, and a liquid carrier. The composition can be formulated to maintain at least 90% available hydrogen peroxide content, and at least 80% viscosity after 4 weeks of storage at 60° C. The composition can be formulated to maintain at least 90% available hydrogen peroxide content, and at least 80% viscosity after 12 months, after 24 months, or at least 36 months of storage at 23° C., or at room temperature. The composition can be formulated as a gel with the composition maintaining greater than 90% available hydrogen peroxide content after 4 weeks storage at 60° C. with no significant loss of gel viscosity (i.e., no greater than 10% loss of gel viscosity). In some aspects, the composition can be formulated as a paste.

The at least one whitening agent can comprise one or more peroxides, such as at least one of a hydrogen peroxide and a urea hydrogen peroxide, and may be present in an amount ranging from 3-50 wt %, or from 6-25 wt % of the composition.

The at least one thickening agent can comprise at least one of a vinylpyrrolidone polymer and a vinylpyrrolidone containing copolymer, and may be present in an amount ranging from 0.1-15 wt % of the composition. In some aspects, the at least one thickening agent can comprise a crosslinked polyvinylpyrrolidone, for example a strongly swellable, lightly to moderately crosslinked polyvinylpyrrolidone, and the at least one thickening agent can be present in an amount ranging from 0.1-10 wt %.

The liquid carrier can comprise one or more of water, glycerin, propylene glycol, polyethylene glycol, sorbitol, ethanol and other suitable liquid carriers. In some aspects, the liquid carrier can comprise water, and the water can be present in an amount between 30-95 wt % of the composition. In some aspects, the liquid carrier can comprise at least one hygroscopic agent selected from the polyol group consisting of glycerin, propylene glycol, polyethylene glycol, polypropylene glycol, and sorbitol. The liquid carrier can be present in an amount between 30-95 wt % of the composition. The hygroscopic agent can be present in an amount between 1-85 wt % of the composition.

Contemplated compositions can also comprise a desensitizing agent, for example, a potassium nitrate, a sodium fluoride, or a mixture thereof. Contemplated compositions can comprise one or more bleaching agent stabilizers, for example, ethylenediaminetetraacetic acid, disodium salt, ethylenediaminetetraacetic acid, tetrasodium salt, ethylenediaminetetraacetic acid, calcium disodium salt, citric acid, etidronic acid, succinic acid, sodium stannate, potassium stannate, sodium phosphate, sodium pyrophosphate, sodium acid pyrophosphate, or a mixture thereof. In some aspects, the bleaching agent stabilizer can be present in an amount of between 0.01-0.5 wt % of the composition. Contemplated compositions can comprise a poly(ethylene oxide)-poly(propylene oxide) block copolymer that is present in an amount of between 1 wt % and 10 wt % of the composition. Contemplated compositions can comprise one or more pH adjusting agents, for example, triethanolamine, sodium hydroxide, potassium hydroxide, or ammonium hydroxide. The one or more pH adjusting agents can be present in an amount of 0.01-5 wt % of the composition. Some compositions can have a pH of at least 6.0, or at least 6.2, or at least 6.5.

In some aspects, a tooth whitening kit is provided, comprising a first part comprising a whitening agent, and a second part comprising an alkaline material, wherein at least one of the first and second parts comprises an effective amount of vinylpyrrolidone polymer or copolymer as a thickening agent. In some aspects, the whitening agent is a peroxide, the second part comprises a strong base dispersed in a stable gel, wherein the strong base comprises at least one of an alkali metal oxide, an alkali metal hydroxide, an alkaline earth metal oxide, and an alkaline earth metal hydroxide. The first part can have an acidic pH and include at least about 25 wt % available hydrogen peroxide. The second part can include at least one strong base dispersed in a stable gel comprising at least one polymeric thickening agent. An apparatus for applying the composition, and instructions for determining a composition application time to achieve a whitening effect may be provided. In some aspects, the first and second parts can be placed in separate chambers of a dual-barrel syringe or other dispenser.

Other advantages and benefits of the disclosed compositions, kits, and systems will be apparent to one of ordinary skill with a review of the following detailed description.

### BRIEF DESCRIPTION

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawing of which:

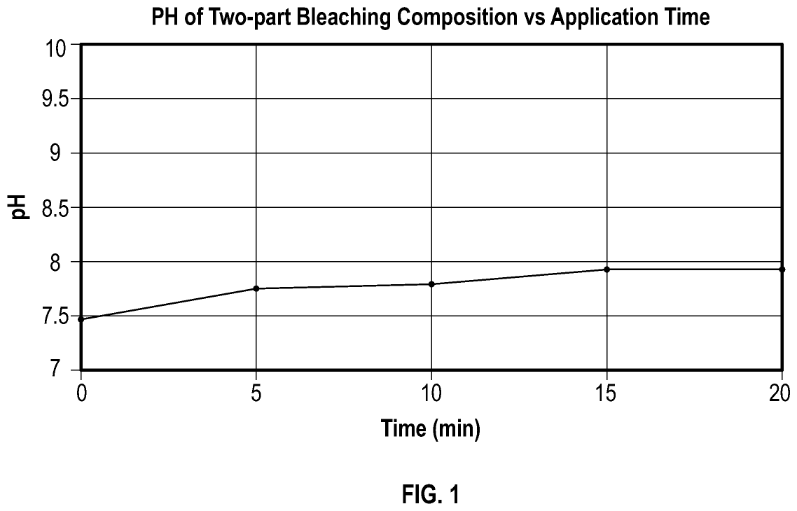

FIG. 1 shows the pH of the mixed composition of Example 7 over time.

### DETAILED DESCRIPTION

After reading this description it will become apparent to one skilled in the art how to implement the disclosed compositions, kits, and systems in various alternative embodiments and alternative applications. However, all the various embodiments of the present disclosure will not be described herein. It is understood that the embodiments presented here are presented by way of an example only, and not limitation. As such, this detailed description of various alternative embodiments should not be construed to limit the scope or breadth of the present disclosure as set forth below.

Many teeth whitening compositions include hydrogen peroxide as an active ingredient, which can be delivered as hydrogen peroxide or carbamide peroxide. Without wishing to be bound by any particular theory, it has been found that hydrogen peroxide is a highly unstable chemical and decomposes into hydrogen cation (H⁺) and perhydroxyl anion (HO₂⁻), which further decomposes to free radical of hydroxyl (HO)● and perhydroxyl (HO₂)● when it reacts with another hydrogen peroxide. Both free radicals are unstable oxidants, which break down the carbon-carbon double bonds that are the source of most stains in the teeth, reduce their light absorption and increase tooth whiteness.

Tooth whitening typically involves an application of peroxide containing composition to the tooth surface for a period of time, for example, between 5 minutes and 8 hour, or between 10 minutes and 2 hours, or between 30 minutes and 1 hour. Tooth whitening products range from over-the-counter (OTC) toothpastes, mouthwashes, whitening strips, whitening pens and whitening syringes, to dentist-prescribed at-home tooth whitening trays or in-office whitening with high concentration of hydrogen peroxide. For the safety and effectiveness purpose, whitening agents are typically formulated into gel or paste forms so they can adhere or remain in contact with tooth surfaces without running to adjacent soft tissue in the oral cavity.

A whitening gel with a pH below 5.5 tends to demineralize tooth enamel, increase tooth surface roughness, reduce its hardness, and reduce fracture resistance. Such gels can also lead to tooth sensitivity and discomfort for the patient or other user. Furthermore, low pH of bleaching gels can reduce their bleaching efficacy by inhibiting the formation of hydroxyl radicals and perhydroxyl radicals, the primary species responsible for the bleaching outcomes. Unfortunately, because hydrogen peroxide has poor stability, commercially available hydrogen peroxide containing dental whitening products typically have a pH below 6.0 in order to maintain adequate shelf life (e.g., two years at room temperature without significant decomposition of the composition).

Carbamide peroxide breaks down in the presence of water to release hydrogen peroxide. Carbamide peroxide is stable when stored in a refrigerator. Although carbamide peroxide containing whitening products can be formulated at a pH of 5.0-6.5 using hygroscopic carriers with acceptable shelf life, their low water content and low peroxide content require greater than 60 minutes of bleaching treatment which can potentially cause tooth sensitivity, tooth dehydration, and the like.

Polyvinylpyrrolidone (PVP) has been used as a thickener for certain tooth whiteners. U.S. Pat. No. 6,500,408 to Chen details a tooth bleach composition that utilizes linear PVP as a thickening agent. It was claimed that the linear PVP must be present in the composition at a concentration of 25% or greater. The linear PVP at high concentrations as described in Chen tends to form sticky or tacky masses, limiting its ability to spread around the teeth for effective bleach and causing gel stringing and roping when dispensed from syringes to the dental whitening tray.

U.S. Pat. No. 8,574,555 to Longo et al describes a specific aqueous tooth whitening composition containing multiple thickening agents, including polyacrylic acid and PVP/hydrogen peroxide complex. The PVP/hydrogen peroxide complex must be present in the composition at a concentration of 10% or greater. Because the PVP/hydrogen peroxide complex prevents ionization of un-neutralized polyacrylic acid backbone, it is required to pre-neutralize polyacrylic acid before combining with PVP/hydrogen peroxide complex.

The present disclosure recognizes the need for compositions, kits, and systems for teeth whitening with improved stability and whitening capabilities. Viewed from another perspective, the present disclosure relates to compositions used to bleach teeth using highly stable oxidizing compositions comprising hydrogen peroxide and a thickening agent comprising lightly to moderately crosslinked vinylpyrrolidone polymer or copolymer. Contemplated compositions thickened with a lightly to moderately crosslinked vinylpyrrolidone polymer or copolymer were surprisingly found to be stable in viscosity and hydrogen peroxide concentration over long term storage at room temperature.

It is desired to have a tooth whitening composition with neutral or near-neutral pH that is stable over an extended period of time when stored at typical room temperature. It also is desired to improve the user experience with contemplated compositions, kits, and systems, such as product handling, spreadability and user safety enhancement.

In some embodiments, a stable one-part aqueous dental bleaching composition having a pH of between 6-7, at least 6, at least 6.2, at least 6.5, or at least 7 is provided. The stability of some contemplated compositions has been proven under accelerated aging conditions that translate into at least a two years shelf life for the composition under typical room temperature storage conditions. Based on applicant's stability testing, for a tooth whitening product that survives for a minimum of 4 weeks at 60° C., it is assumed that it would survive on the open market for a minimum of two years under room temperature storage conditions.

In some aspects, contemplated stable tooth whitening compositions can comprise a bleaching agent, a polymeric thickening agent, a liquid carrier, and a pH adjusting agent. The pH of the tooth bleaching composition during the bleaching process can be in a range of pH 6.0-9.0.

In some aspects, a one-part tooth whitening system is provided comprising a bleaching agent, a polymeric thickening agent, a liquid carrier, and a pH adjusting agent. The one-part bleaching system can have a pH above 6.0 and can be storage stable in hydrogen peroxide concentration and viscosity.

In another aspect, the invention provides a stable tooth bleaching composition with improved performance. The improved performance can include, among other things, improved stability, handling, consistency, spreadability and application control.

In accordance with another aspect, the disclosure relates to a polymeric thickener, a crosslinked vinylpyrrolidone polymer or copolymer.

One-Part Teeth Whitening Systems

The present disclosure provides a one-part tooth bleaching composition that is stable at typical room temperature storage conditions for an extended period of time. The compositions can comprise a hydrogen peroxide bleaching agent that releases peroxide to the tooth surface for a desired bleaching effect without appreciable decomposition when stored in plastic syringes, pens, or other suitable containers.

In some aspects, a one-part tooth whitening composition is provided, comprising at least one whitening or bleaching agent, at least one thickening agent, and a liquid carrier. A basic agent, a desensitizing agent, a flavorant, a combination thereof, or any other suitable ingredients may also be included.

The composition can be formulated to maintain at least 90% available hydrogen peroxide content, and at least 80% viscosity after 4 weeks of storage at 60° C. The composition can be formulated to maintain at least 90% available hydrogen peroxide content, and at least 80% viscosity after 12 months, after 24 months, or at least 36 months of storage at 23° C., or at room temperature. The composition can be formulated as a gel with the composition maintaining greater than 90% available hydrogen peroxide content after 4 weeks storage at 60° C. with no significant loss of gel viscosity.

Whitening or Bleaching Agent

Tooth whitening compositions typically include a form of tooth whitening active, such as hydrogen peroxide or carbamide peroxide, a hydrogen peroxide precursor. Suitable whitening or bleaching agents for compositions, kits and systems of the disclosure include any material that whitens a tooth surface to which it is applied, for example, a peroxide compound. A peroxide is an oxidizing compound with the structure R—O—O—R, where R=any element. Peroxide compounds include peroxides and hydroperoxides, such as hydrogen peroxide, peroxides of alkali and alkaline earth metals, organic peroxy compounds, peroxy acids, pharmaceutically-acceptable salts thereof, and mixtures thereof. Peroxides of alkali and alkaline earth metals include lithium peroxide, potassium peroxide, sodium peroxide, magnesium peroxide, calcium peroxide, barium peroxide, and mixtures thereof. Organic peroxy compounds include carbamide peroxide (also known as urea hydrogen peroxide), glyceryl hydrogen peroxide, alkyl hydrogen peroxides, dialkyl peroxides, alkyl peroxy acids, peroxy esters, diacyl peroxides, benzoyl peroxide, and monoperoxy phthalate, and mixtures thereof. Peroxy acids and their salts include organic peroxy acids such as alkyl peroxy acids, and monoperoxyphthalate and mixtures thereof, as well as inorganic peroxy acid salts such as persulfate, dipersulfate, percarbonate, perphosphate, perborate and persilicate salts of alkali and alkaline earth metals such as lithium, potassium, sodium, magnesium, calcium and barium, and mixtures thereof.

In some contemplated embodiments, hydrogen peroxide or urea hydrogen peroxide is preferred as a whitening agent, and can be present in one-part bleaching systems in an amount from about 3-50 wt %, about 5-25 wt %, about 6-15 wt %, or about 9-15 wt % of the composition. In some aspects, the hydrogen peroxide composes a 30-50 wt % hydrogen peroxide solution and the hydrogen peroxide solution comprises between 10-50 wt % of the composition, with the hydrogen peroxide comprising between 3-25 wt % of the composition.

Thickening Agent

Compositions, kits and systems of the disclosure can include a thickening agent to yield a composition with a desired viscosity. All orally acceptable thickening agents are contemplated. In some preferred aspects, the one or more thickening agents comprise at least one of a vinylpyrrolidone polymer or copolymer, and may be present in an amount ranging from 0.1-15 wt %, or about 1-10 wt % of the composition. In some aspects, the at least one thickening agent can comprise a crosslinked polyvinylpyrrolidone (PVP), for example, a lightly to moderately crosslinked PVP, and the at least one thickening agent can be present in an amount ranging from 0.1-15 wt %, or about 1-10 wt %. Applicant surprisingly discovered that a lower concentration of crosslinked PVP could be used, while having a clear, flowable and homogeneous gel formulation that is shelf stable in viscosity and hydrogen peroxide concentration over long term storage at room temperature. In some aspects, the at least one thickening agent comprises a strongly swellable crosslinked PVP, for example, a lightly to moderately crosslinked PVP. The lightly to moderately crosslinked PVP does not refer to swellable but water insoluble crosslinked PVP, such as the type sold under the trade name Kollidon® by BASF AG. In some embodiments, the compositions, kits, and systems can comprise one or more of a FlexiThix™ polymer (INCI name polyvinylpyrrolidone [PVP], Ashland Specialty, Wayne, NJ), ACP-1120, ACP-1179, and ACP-1180, available from Ashland INC (Wayne, NJ) as a thickening agent.

In some aspects, the at least one thickening agent comprises a lightly to moderately crosslinked PVP in a range of about 0.1-20% by weight of the tooth whitening composition. In some aspects, the at least one thickening agent comprises a lightly to moderately crosslinked PVP in a range of about 2-12% by weight of the tooth whitening composition. In some aspects, the at least one thickening agent comprises a lightly to moderately crosslinked PVP in a range of about 4-10% by weight of the tooth whitening composition. In some aspects, the at least one thickening agent comprises a lightly to moderately crosslinked PVP in a range of about 4-9% by weight of the tooth whitening composition. It should be appreciated that the concentration of PVP may be adjusted to achieve a desired viscosity, stickiness, or other physical properties.

Lightly to moderately crosslinked PVP is a nonionic polymer and advantageously is easily dispersed in aqueous, glycols, alcohol, and glycerin-based systems. It does not require neutralization and is highly salt tolerant. Unlike un-crosslinked PVP which produces a sticky or tacky bleaching gel, the compositions of the present invention can be made less tacky with lightly to moderately crosslinked PVP if desired, particularly if it is directly applied from a pen.

The lightly to moderately crosslinked vinylpyrrolidone polymer can comprise strongly swellable, moderately crosslinked PVP polymer powders characterized by an aqueous gel volume of about 15-150 ml/g of polymer and a Brookfield viscosity in 5% aqueous solution of at least 10,000 cps, as described in U.S. Pat. Nos. 5,073,614 and 5,139,770. It should be appreciated that gel volume is a measure of the swelling property of the crosslinked polymer, which can be determined by adding 1 g of the polymer to a suitable container filled with water, shaking the mixture and allowing it to stand at room temperature for 3 days. The volume of the gel produced in water is measured and taken as the gel volume.

In some aspects, the lightly to moderately crosslinked vinylpyrrolidone polymer can comprise strongly swellable, moderately crosslinked PVP polymer powders characterized by an aqueous gel volume of about 25-75, or 30-60 ml/g of polymer and a Brookfield viscosity in 5% aqueous solution of at least 15,000 cps, at least 20,000 cps, between 20,000-50,000 cps. In some aspects, the moderately crosslinked vinylpyrrolidone polymer can be prepared directly by precipitation polymerization of monomers consisting essentially of vinyl pyrrolidone in an organic solvent in the presence of a crosslinking agent (e.g., one or more of N,N′-divinylimidazolidone, pentaerythritol triallyl ether, triallyl-1,3,5-triazine-2,4,6(1H,3H,5H)-trione and 2,4,6-triallyloxy-1,3,5-triazine, or any other suitable crosslinking agent) in the amount of about 0.2 to about 1% by weight of vinyl pyrrolidone and a free radical polymerization initiator. In some aspects, the moderately crosslinked vinylpyrrolidone polymer can be prepared directly by precipitation polymerization of monomers consisting essentially of vinyl pyrrolidone in an organic solvent in the presence of a crosslinking agent in the amount of about 0.25-0.8%, or about 0.35-0.6% by weight of vinyl pyrrolidone and a free radical polymerization initiator (e.g., acyl peroxides such as diacetyl peroxide, dibenzoyl peroxide and dilauryl peroxide; peresters such as t-butylperoxy pivalate, tert-butyl peroxy-2-ethylhexanoate; peroxides such as di-tert-butyl peroxide; percarbonates such as dicyclohexyl peroxydicarbonate; and azo compounds such as 2,2′-azobis(isobutyronitrile), 2,2′-azobis(2,4-dimethylvaleronitrile), 1,1′-azobis(cyanocyclohexane), and 2,2′-azobis(methylbutyronitrile), or any other suitable initiator).

In some aspects, the strongly swellable, lightly to moderately crosslinked polyvinylpyrrolidone can be made directly as fine, white powders comprising or consisting essentially of precipitation polymerizing vinyl pyrrolidone in the presence of a crosslinking agent and a free radical polymerization initiator in an organic solvent (e.g., an aliphatic hydrocarbon which is a C₃-C₁₀ saturated hydrocarbon, branched or unbranched, cyclic or acyclic, or mixtures thereof, or any other suitable organic solvent) at a polymerization temperature of about 40° C. to 150° C., or between about 60° C. to 70° C. In some aspects, the crosslinking agent can be present in an amount of about 0.2 to 1%, between 0.25-0.8%, or between 0.35-0.6% by weight of the vinyl pyrrolidone monomer. Pressure can be kept at atmospheric pressure, although higher and lower pressures may be used as well.

In some aspects, the at least one thickening agent comprises a vinylpyrrolidone copolymer of an ammonium or alkali salt of acryloyldimethyltaurate and ethylene, acryloyldimethyltaurate and styrene or acryloyldimethyltaurate and vinylpyrrolidone, preferred are copolymers or mixtures of copolymers based on acryloyldimethyltaurate and least one ethylenically unsaturated monomer selected from vinylpyrrolidone, styrene and vinylpolyethylene oxide. Available examples include but are not limited to Ultrathix P-100, and Lubrajel from Ashland Specialty (Wayne, NJ). Aristoflex AVC, Aristoflex AVS and Aristoflex HMB from Clariant International LTD. In some aspects, the at least one thickening agent is present in a range of about 0.1-20% by weight of the tooth whitening composition. In some aspects, the at least one thickening agent is present in a range of about 2-12% by weight of the tooth whitening composition. In some aspects, the at least one thickening agent is present in a range of about 4-10% by weight of the tooth whitening composition. In some aspects, the at least one thickening agent is present in a range of about 4-9% by weight of the tooth whitening composition.

Liquid Carrier

The liquid carrier(s) or solvent(s) can comprise one or more of water, glycerin, propylene glycol, polyethylene glycol, sorbitol, ethanol and other suitable liquid carriers. In some aspects, the liquid carrier can comprise water, and the water can be present in an amount, for example, between 15-30 wt %, 15-50 wt %, or 30-95 wt % of the composition. In some aspects, the liquid carrier can comprise at least one hygroscopic agent selected from the polyol group consisting of glycerin, propylene glycol, polyethylene glycol, polypropylene glycol, and sorbitol. The hygroscopic agent can be present in an amount, for example, between 1-85 wt %, between 30-70 wt %, or between 35-55 wt % of the composition. The liquid carrier can be present in an amount, for example, between 30-95 wt %, between 30-85 wt %, between 50-85 wt %, or between 50-80 wt % of the composition. In some embodiments, the liquid carriers comprise water and a hygroscopic agent, wherein the water can be present in an amount between, for example, 15-25 wt % of the composition, and wherein the hygroscopic agent is present in an amount between, for example, 35-55 wt %. In some aspects, the solvent is selected and present in an amount sufficient to form a gel with suitable viscosity for teeth whitening purposes.

Basic Agent

To avoid tooth etching and improve bleaching efficiency, it is preferable to add a pH adjuster, or more specifically, a base in order to increase the pH of tooth bleaching compositions to a pH range of about 5 to 7, or in some preferred aspects, a pH range of about 6 to 7, or 6.2 to 7.5. Compositions, kits, and systems of the disclosure can comprise any suitable pH adjuster, including, for example, a triethanolamine, sodium hydroxide, potassium hydroxide, ammonium hydroxide, or a combination thereof. In some embodiments, an aqueous potassium hydroxide can be a preferred pH adjuster. The pH adjuster may be present in any suitable amount in a composition, kit, or system. For example, the pH adjusters can be included in a range from about 1.0% to about 10.0% by weight of the composition, or in a range from about 2% to about 8% by weight of the composition.

Other Components

In some embodiments of the disclosed compositions, kits, and systems, it may be desirable to include other components such as stabilizers, desensitizing agents, flavoring agents, sweeteners, anticariogenic agents, and antimicrobial agents.

The tooth bleaching compositions, kits, and systems can include a poly(ethylene oxide)-poly(propylene oxide) block copolymer or a mixture of these copolymers to adjust the thixotropic properties of the composition or parts of a kit. In some aspects, a composition, kit or system of the disclosure can include a mixture of poly(ethylene oxide)-poly(propylene oxide)-poly(ethylene oxide) with a molecular weight range from 5000 g/mol to 15000 g/mol. An example includes a mixture of POLOXAMER 407 and POLOXAMER 188, also known as PLURONIC F127 and PLURONIC F68, respectively, which can be present in an amount of, for example, about 3-10 wt % of the composition.

The tooth bleaching compositions, kits, and systems can include a bleaching agent stabilizer, including, for example, ion scavengers, such as ethylenediaminetetraacetic acid, disodium salt, ethylenediaminetetraacetic acid, tetrasodium salt, ethylenediaminetetraacetic acid, calcium disodium salt, citric acid, etidronic acid, succinic acid, sodium stannate, potassium stannate, sodium phosphate, sodium pyrophosphate, sodium acid pyrophosphate, or a combination or mixture thereof. In some aspects, the bleaching agent stabilizers can be included in an amount in a range from about 0% to about 1% by weight of the whitening composition, or from about 0.01% to about 0.5% by weight of the whitening composition.

The tooth bleaching compositions, kits, and systems can include one or more desensitizers (e.g., potassium nitrate, sodium fluoride) to alleviate or prevent tooth sensitivity of patients or other users. One or more desensitizers may be present in the composition in an amount from about 0.1-3%, or from about 0.3-0.8% by weight of the composition.

The tooth bleaching compositions, kits, and systems can include one or more flavorants or sweeteners (e.g., peppermint, spearmint, corn mint, wintergreen, green tea, orange, lemon).

The tooth bleaching compositions, kits, and systems can include any additional ingredients suitable for human administration, for example, moisturizing agents, solubilizing agents, flow agents, preservatives, colors, flavors, and the like.

The tooth whitening composition may be stored in syringes, pens, capsules or any suitable containers. The tooth whitening composition can be applied to teeth by a brush, a swab, a sponge, or other suitable applicator.

Multi-Part Teeth Whitening Kits and Systems

In some aspects, a multi-part tooth whitening kit is provided, comprising a first part comprising a whitening agent, and a second part comprising an alkaline material, wherein at least one of the first and second parts comprises an effective amount of lightly to moderately crosslinked vinylpyrrolidone polymer or vinylpyrrolidone copolymer as a thickening agent. In some aspects, the whitening agent is a peroxide, the second part comprises a strong base dispersed in a stable gel, wherein the strong base comprises at least one of an alkali metal oxide, an alkali metal hydroxide, an alkaline earth metal oxide, and an alkaline earth metal hydroxide. The first part can have an acidic pH and include at least about 25 wt %, at least 30 wt %, at least 35 wt %, at least 40 wt %, or at least 45 wt % available hydrogen peroxide. The second part can include at least one strong base dispersed in a stable gel comprising at least one polymeric thickening agent. An apparatus for applying the composition, and instructions for determining a composition application time to achieve a whitening effect may be provided. In some aspects, the first and second parts can be placed in separate chambers of a dual-barrel syringe or other dispenser.

Viewed from another perspective, a multi-part bleaching system that comprises a first whitening agent component or formulation and a second strong base component or formulation. In some aspects, a user, whether at home or in a dental office, can mix the two components in a desired ratio prior to use. The desired ratios can comprise, for example, from about 1:5 to about 10:1 (whitening agent component:strong base component), from about 1:1 to about 10:1 (whitening agent component:strong base component), from about 1:5 to about 5:1 (whitening agent component:strong base component), from about 1:1 to about 5:1 (whitening agent component:strong base component), from about 1:1 to about 5:1 (strong base component:whitening agent component), or any other suitable ratio.

Whitening Agent Component (Peroxide Part)

The first part of the multi-part tooth whitening kit or system can include a dental bleaching agent and a polymeric thickener. In some aspects, a preferred whitening agent is hydrogen peroxide. Other whitening agents, such as carbamide peroxide, perborates are also contemplated. In order to generate strong bleach strength which is generally required for in-office whitening, high concentrations of aqueous hydrogen peroxide can be present in the composition. The amount of aqueous hydrogen peroxide (e.g. 50 wt % solution of hydrogen peroxide, 30-50 wt % solution of hydrogen peroxide), may be present in a range from about 50% to about 99% by weight of the composition in the peroxide part, preferably in a range from about 60% to about 96%, or from about 80-96 wt % by weight of composition in the peroxide part. In some aspects, the whitening agent (e.g., H₂O₂) can be present in a range of about 25-50%, or about 30-48%, or about 40-48% by weight of the composition in the peroxide part.

The peroxide part of the multi-part tooth whitening kit or system can also include at least one thickener in an amount from about 0.1-10%, 0.1 to about 8%, from about 5 to about 10%, from about 0.1 to about 15%, from about 1% to about 15%, or from about 6% to about 12% by weight of the composition. All suitable thickening agents are contemplated, including, for example, a lightly to moderately crosslinked vinylpyrrolidone polymer, a vinylpyrrolidone containing copolymer, FlexiThix™, or Aristoflex.

Other agents such as fumed silica, stabilizers, dyes, and flavorants may also be included as desired. Contemplated peroxide parts can comprise a desensitizing agent, for example, a potassium nitrate, a sodium fluoride, or a mixture thereof. Contemplated peroxide parts can comprise one or more bleaching agent stabilizers, for example, ethylenediaminetetraacetic acid, disodium salt, ethylenediaminetetraacetic acid, tetrasodium salt, ethylenediaminetetraacetic acid, calcium disodium salt, citric acid, etidronic acid, succinic acid, sodium stannate, potassium stannate, sodium phosphate, sodium pyrophosphate, sodium acid pyrophosphate, or a mixture thereof. In some aspects, the bleaching agent stabilizer can be present in an amount of between 0.1-2 wt %, between 0.1-1 wt %, or between 0.01-0.5 wt % of the composition. Contemplated peroxide parts can comprise a poly(ethylene oxide)-poly(propylene oxide) block copolymer that is present in an amount of between 1 wt % and 10 wt % of the composition.

In some aspects, the peroxide part can have a pH below 7, more preferably below 6, below 5, below 4, below 3, between 1-4, or between 1-3 for stability of the whitening agent.

Strong Base Component (Activation Part)

The second part of the multi-part tooth whitening kit or system can comprise a strong base gel which can include a liquid carrier, a strong base, and a polymeric thickener. It may also include a desensitizer, a flavorant, a dye, as desired. The strong base gel can be used to increase the pH of the tooth whitening kit or system to a desired value when mix with the peroxide part.

The one or more liquid carriers can comprise water, ethanol and liquid polyols such as propylene glycol and polyethylene glycol, or any other suitable carrier(s). The liquid carriers can be present in an amount, for example, from about 10-99 wt %, about 50-95 wt %, or from about 60-90 wt % of the activation part. In some aspects, the one or more liquid carriers comprise water and polyethylene glycol, wherein water can be present in an amount, for example, from about 10% to about 95% by weight, from about 20% to about 90% by weight, from about 30% to about 70% by weight, from about 40% to about 60% by weight, from about 10% to about 50% by weight, from about 10% to about 40% by weight, from about 30% to about 50% by weight of the activation part, and wherein polyethylene glycol can be present in an amount, for example, from about 10% to about 60% by weight, preferably in a range from about 40% to about 60%, or from about 35% to about 50% by weight of the activation part. The polyethylene glycol can have a molecular weight of, for example, about 200 to about 600 g/mol.

Strong bases suitable for this invention include hydroxides and carbonates of alkali which dissolve in the liquid carrier. Examples of strong bases includes sodium hydroxide and potassium hydroxide. In some aspects, the strong bases can be present in the activation part in an amount in a range from about 1% to about 4% by the weight, or in a range from about 1.5% to about 4% by weight.

The activation part of the multi-part tooth whitening kit or system can also include at least one thickener in an amount from about 0.1-10% by weight, 0.1 to about 8% by weight, 0.1 to about 15% by weight of the composition, 1% to about 15% by weight of the composition, and in some embodiments in a range from about 6% to about 12%, from about 3% to about 8%, or from about 4% to about 6% by weight of the activation part. All suitable thickening agents are contemplated, including, for example, a vinylpyrrolidone polymer, a vinylpyrrolidone containing copolymer, or a crosslinked polyvinylpyrrolidone. In some aspects, a crosslinked PVP, for example, a lightly to moderately crosslinked PVP, which can be capable of thickening in broad pH ranges, can be preferred.

Contemplated activation parts can also comprise a desensitizing agent, for example, a potassium nitrate, a sodium fluoride, or a mixture thereof. Contemplated activation parts can comprise a poly(ethylene oxide)-poly(propylene oxide) block copolymer that is present in an amount of between 1 wt % and 10 wt %, or between 4 wt % and 8 wt % of the activation part. Contemplated activation parts can comprise one or more pH adjusting agents, for example, triethanolamine, sodium hydroxide, potassium hydroxide, or ammonium hydroxide. The one or more pH adjusting agents can be present in an amount of, for example, 0.01-10 wt % or 0.01-5 wt % of the activation part. Some activation part can have a pH of, for example, at least 10, or at least 12, or at least 14.

The peroxide part or the activation part can include any additional ingredients suitable for human administration, for example, moisturizing agents, solubilizing agents, flow agents, preservatives, colors, flavors, and the like. In some aspects, the peroxide part and the activation part can include different dyes such that the parts have different colors from one another for easy identification.

Mixed Composition from Peroxide Part and Activation Part

The peroxide part and activation part can be stored separately. For example, a tooth whitening kit that includes a peroxide part and an activation part in separate containers, or a dual barrel syringe that stores a peroxide part and an activation part separately. The peroxide part and activation part may be mixed in any suitable mode prior to use. For example, auto mixed with a dual barrel mixing tip or hand mixed two parts from separate containers.

The peroxide part and activation part may be mixed in any desired ratios for example, from about 1:5 to about 10:1 (peroxide part:activation part), from about 1:1 to about 10:1 (peroxide part:activation part), from about 1:5 to about 5:1 (peroxide part:activation part), from about 1:1 to about 5:1 (peroxide part:activation part), from about 1:1 to about 5:1 (activation part:peroxide part), from about 3.5:1 to about 4.5:1 (peroxide part:activation part), about 4:1 (peroxide part:activation part), or any other suitable ratio. The pH of the mixed composition, which may be a gel, can be in the range of 5 to 9, and in some preferred aspects, a range of 7 to 9. Once the parts are mixed, the mixed tooth bleaching composition may be applied to at least one tooth using any known application method or applicator. The kit can comprise a brush, a swab, a sponge, or other suitable applicator for applying or spreading the combined parts onto a user's teeth. The mixed whitening composition may remain on the tooth for any suitable period of time, for example, a period of time ranging from about 1 minute to about 30 minutes, from about 5 minutes to about 25 minutes, or from 8 minutes to about 20 minutes.

### EXAMPLES

The examples below are presented for illustrative purposes. For each of the single-part tooth whitening composition examples set forth in Table 1, all powdered stabilizer, salts and powdered thickening agents were weighed in a plastic mixing cup. Deionized water and aqueous hydrogen peroxide were then added. The blends were mixed by a Speedmixer DAC 150 FV from FlackTek. When all powdered ingredients were fully dissolved, polyols, pH adjusting agent and other ingredients were added and thoroughly mixed. Each of the tooth whitening compositions were deaerated and then packed in 3 cc clear LDPE Luer-lock syringes for stability evaluation.

The finished compositions were tested for hydrogen peroxide content, viscosity, and pH.

Hydrogen peroxide determination. Hydrogen peroxide concentration, expressed as weight percentage of the total gel composition weight, was determined by a standard iodometric titration (Solvay technical data sheet, determination of hydrogen peroxide concentration).

Viscosity determination. The viscosity of the whitening compositions were determined by using a Brookfield R/S Plus viscometer with cone-plate attachment. The spindle RCT-50-1 was used at spindle speed of 100 rpm at 25° C.

pH determination. The pH was measured by a pH meter of the Accumet Model AB250 (Fisher Scientific) with a flat pH probe (Thermo Scientific™ 9135APWP). The pH meter was calibrated with standard pH buffer solutions at pH 4 and pH 7 before measurement. The pH value of the whitening composition was obtained by inserting the pH probe into the whitening gel and reading the pH value when it was stabilized.

TABLE 1
Compositions of Representative Tooth Whitening Gels

| | Example 1 | Example 2 | Example 3 | Example 4 | Example 5 | Example 6 |
|---|---|---|---|---|---|---|
| Ingredient | wt % | wt % | wt % | wt % | wt % | wt % |
| Water | 19.6 | 19.6 | 25.1 | 21.2 | 25.0 | 20.0 |
| Polyethylene | 45.4 | 40.5 | 49.3 | 50.0 | 39.8 | 38.8 |
| glycol | | | | | | |
| Propylene | — | — | — | — | — | 15.0 |
| glycol | | | | | | |
| Hydrogen | 19.6 | 19.6 | 16.7 | 19.8 | 28.0 | 18.0 |
| peroxide | | | | | | |
| (50%) | | | | | | |
| FlexiThix | 8.8 | 8.8 | 7.5 | — | 5.0 | 6.0 |
| Aristoflex | — | — | — | 2.0 | — | — |
| Poloxamer | 4.9 | 8.8 | — | 5 | — | — |
| Disodium | 0.2 | 0.20 | 0.2 | 0.2 | 0.2 | 0.2 |
| EDTA | | | | | | |
| KNO₃ | 0.5 | 0.5 | 0.4 | — | 0.5 | 0.5 |
| Potassium | To pH 5.6 | To pH 6.3 | To pH 5.4 | To pH 5.8 | to pH 6.3 | to pH 7.0 |
| hydroxide | | | | | | |
| (0.5N) | | | | | | |
| Flavor | >0.05 | >0.05 | >0.05 | >0.05 | >0.05 | >0.05 |
| Total | 100 | 100 | 100 | 100 | 100 | 100 |

Each of the compositions above were viscous but not stringy. They could be easily dispensed from a syringe. The compositions exhibited high yield stress which prevented the compositions from running off.

A two-part tooth whitening system was formulated which includes the components in Part A and Part B as set forth in Table 2, with Part A and Part B being two separate parts that are combined prior to use to form a whitening composition. The kit or system was designed to combine Part A and Part B in a volume ratio of about 4 to 1 (or between 3.5:1 and 4.5:1 Part A to Part B) to form an activated bleaching composition prior to use.

### Two-Part Tooth Bleaching Composition

### Example 7

TABLE 2

| | Part A | Part B |
|---|---|---|
| Ingredient | wt (%) | wt (%) |
| Deionized water | — | 42.00 |
| Polyethylene glycol | — | 45.00 |
| Hydrogen peroxide 50% | 91.00 | — |
| FlexiThix | 9.00 | 5.00 |
| NaOH | — | to |
| | | pH >14 |
| KNO₃ | — | 6.00 |
| Dye | — | <0.1 |
| Total | 100 | 100 |

Each of Parts A and B were stable viscous gels but not tacky. Parts A and B were mixed and formed a mixed composition that included the foregoing components in a volume ratio of 4 to 1. The pH of the immediately mixed composition was above 7.4 and was increased to 7.9 in 20 minutes after mixing, as shown in FIG. 1. The mixed composition remained viscous but not tacky. It was easily dispensed on tooth.

Stability of Hydrogen Peroxide at Accelerated Aging

The aging stability of tooth bleaching composition is very important to the product's performance and efficacy. Table 3 shows the properties of the tooth bleaching compositions of Examples 1, 2 and 3 stored at 60° C., as well as Example 4 stored at 50° C. It was demonstrated that less than 10% of the initial hydrogen peroxide content was degraded for Examples 1, 2, 3 and 4 after the storage period. There was no significant change in viscosity of Example 1. The viscosity of Example 2, 3 and 4 reduced 9%, 13% and 16% respectively when compared with the baseline.

TABLE 3
Material Properties after storage at 50° C. and 60° C.

| Name | Aging condition | HP % | Viscosity (cP) |
|---|---|---|---|
| Example 1 | Original | 9.8% | 6079 |
| | 8 wk at 60° C. | 9.4% | 6209 |
| Example 2 | Original | 9.9% | 7154 |
| | 8 wk at 60° C. | 9.0% | 6497 |
| Example 3 | Original | 8.3% | 6970 |
| | 6 wk at 60° C. | 7.6% | 6054 |
| Example 4 | Original | 10.9% | 9968 |
| | 10 wk at 50° C. | 10.0% | 8361 |

Accelerated aging testing is commonly used to justify shelf life of new products when room temperature stability tests would delay its introduction to market. Typically, a whitening product stored at 60° C. for 4 weeks can be reliably projected to be stable at room temperature (23° C.) for at least 24 months. As shown in Table 4, all examples from 1 to 6 survived for an excess of 4 weeks at 60° C. with less than 10% of loss of hydrogen peroxide content.

TABLE 4
Hydrogen peroxide (HP) content after storage at 60° C. for 28 days

| | Example 1 | Example 2 | Example 3 | Example 4 | Example 5 | Example 6 |
|---|---|---|---|---|---|---|
| HP % (original) | 9.8% | 9.8% | 8.3% | 9.9% | 14.0% | 9.0% |
| HP % (28 d at 60° C.) | 9.6% | 9.4% | 7.7% | 9.7% | 13.0% | 8.7% |
| HP retain rate (%) | 98.0% | 95.9% | 92.8% | 98% | 92.9% | 96.7% |

Thus, specific examples of teeth whitening compositions, kits, and systems have been disclosed. It should be apparent, however, to those skilled in the art that many more modifications besides those already described are possible without departing from the inventive concepts herein. While examples and variations of the many aspects of the invention have been disclosed and described herein, such disclosure is provided for purposes of explanation and illustration only. Thus, various changes and modifications may be made without departing from the scope of the claims.

The terminology used herein is for the purpose of describing particular cases only and is not intended to be limiting. The below terms are discussed to illustrate meanings of the terms as used in this specification, in addition to the understanding of these terms by those of skill in the art. As used herein and in the appended claims, the singular forms “a,” “an,” and “the” include plural referents unless the context clearly dictates otherwise. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

Reference throughout this specification to “an embodiment” or “an implementation” means that a particular feature, structure, or characteristic described in connection with the embodiment is included in at least one embodiment or implementation. Thus, appearances of the phrases “in an embodiment” in various places throughout this specification are not necessarily all referring to the same embodiment or a single exclusive embodiment. Furthermore, the particular features, structures, or characteristics described herein may be combined in any suitable manner in one or more embodiments or one or more implementations.

Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refer to at least one of something selected from the group consisting of A, B, C . . . and N, the text should be interpreted as requiring only one element from the group, not A plus N, or B plus N, etc.

The word “exemplary” is used herein to mean “serving as an example, instance, or illustration.” Any aspect described herein as “exemplary” is not necessarily to be construed as preferred or advantageous over other aspects. Unless specifically stated otherwise, the term “some” refers to one or more.

Unless the context dictates the contrary, all ranges set forth herein should be interpreted as being inclusive of their endpoints and open-ended ranges should be interpreted to include only commercially practical values. Similarly, all lists of values should be considered as inclusive of intermediate values unless the context indicates the contrary. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating un-recited number may be a number which, in the context in which it is presented, provides the substantial equivalent of the specifically recited number.

Combinations such as “at least one of A, B, or C,” “one or more of A, B, or C,” “at least one of A, B, and C,” “one or more of A, B, and C,” and “A, B, C, or any combination thereof” include any combination of A, B, and/or C, and may include multiples of A, multiples of B, or multiples of C. Specifically, combinations such as “at least one of A, B, or C,” “one or more of A, B, or C,” “at least one of A, B, and C,” “one or more of A, B, and C,” and “A, B, C, or any combination thereof” may be A only, B only, C only, A and B, A and C, B and C, or A and B and C, where any such combinations may contain one or more member or members of A, B, or C.

All structural and functional equivalents to the components of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

Although the disclosure has been described with reference to the above examples, it will be understood that modifications and variations are encompassed within the spirit and scope of the disclosure. Accordingly, the disclosure is limited only by the following claims.

What is claimed is:

1. A tooth whitening kit, comprising:
a first part comprising a whitening agent, wherein the whitening agent comprises hydrogen peroxide and is present in an amount of at least 35 wt % of the first part; and
a second part comprising an alkaline material;
wherein at least one of the first and second parts comprises a crosslinked polyvinylpyrrolidone as a thickening agent in an amount of between 6-12 wt %; and
wherein combining the first part and the second part forms an activated tooth whitening composition with a pH of between 7 and 9.

2. The kit of claim 1, wherein the whitening agent is a peroxide, and wherein the second part comprises a strong base dispersed in a stable gel, wherein the strong base comprises at least one of an alkali metal oxide, an alkali metal hydroxide, an alkaline earth metal oxide, and an alkaline earth metal hydroxide.

3. The tooth whitening kit of claim 1, wherein at least one of the first part and second part further comprises a poly(ethylene oxide)-poly(propylene oxide) block copolymer that is present in an amount of between 1 wt % and 10 wt % of the composition.

4. The tooth whitening kit of claim 2, wherein the strong base comprises sodium hydroxide.

5. The tooth whitening kit of claim 1, wherein a pH of the activated tooth whitening composition increases during the first twenty minutes after the activated tooth whitening composition is formed.

6. The tooth whitening kit of claim 1, wherein each of the first and second parts comprises the crosslinked polyvinylpyrrolidone as the thickening agent.

7. The tooth whitening kit of claim 1, further comprising a dual barrel syringe that stores the first part and second part separately.

8. The tooth whitening kit of claim 7, wherein the dual barrel syringe comprises a mixing tip.

9. The tooth whitening kit of claim 1, wherein the second part comprises one or more liquid carriers, and wherein the one or more liquid carriers are present in an amount of about 50-95 wt % of the second part.

10. The tooth whitening kit of claim 1, wherein the second part has a pH of at least 12.

11. The tooth whitening kit of claim 1, wherein the thickening agent is present in the first part in an amount from about 5 to about 10 wt %.

12. The tooth whitening kit of claim 1, wherein the kit is at least one of an at-home kit and an in-office kit.

13. The tooth whitening kit of claim 1, wherein each of the first and second parts comprises the crosslinked polyvinylpyrrolidone as a thickening agent, and wherein each of the first and second parts comprises a viscous gel.

14. The tooth whitening kit of claim 1, wherein the crosslinked polyvinylpyrrolidone comprises a lightly to moderately crosslinked polyvinylpyrrolidone.

15. The tooth whitening kit of claim 1, wherein the whitening agent comprises hydrogen peroxide.
